(19) United States
(12) Patent Application Publication
(10) Pub. No.: US 2024/0259376 A1
(43) Pub. Date: Aug. 1, 2024

(54) METHODS AND SYSTEMS FOR ASSIMILATING CLIENT-FACING DATA AND CLIENT INSTRUCTIONS FOR A BIOMANUFACTURING SYSTEM

(71) Applicant: Culture Biosciences, Inc., South San Francisco, CA (US)

(72) Inventors: Tiffany Huang, South San Francisco, CA (US); Satshabad Khalsa, South San Francisco, CA (US); Ivey Topaz, South San Francisco, CA (US); Tara Iritsky, South San Francisco, CA (US); Garret Meier, South San Francisco, CA (US); Casey Chance, South San Francisco, CA (US)

(21) Appl. No.: 18/419,273

(22) Filed: Jan. 22, 2024

Related U.S. Application Data
(60) Provisional application No. 63/441,577, filed on Jan. 27, 2023.

(51) Int. Cl. H04L 9/40; C12M 1/107; H04L 67/10

(52) U.S. Cl. CPC H04L 63/10; C12M 21/04; H04L 67/10

(57) ABSTRACT

Systems and methods described herein provide a data presentation, assimilation and monitoring system for a biomanufacturing system utilizing an array of bioreactors for conducting biomanufacturing experiments. In embodiments, the experiments may be controlled by recipes that can be created and altered by credentialed users. During experiments, credentialed users may monitor operational parameters of the bioreactors conducting experiments as well as batch and group parameters across multiple bioreactors. Further, credentialed users may alter recipes to the exclusion of other credentialed users while editing. Further yet, specific checklists may be utilized to ensure that correct and suited equipment (such as bioreactors and experiment bays) are matched with an experiment plan.

### CLAIM OF PRIORITY

This application claims the benefit of U.S. Provisional Application No. 63/441,577 entitled “METHODS AND SYSTEMS FOR ASSIMILATING CLIENT-FACING DATA AND CLIENT INSTRUCTIONS FOR A FERMENTATION SYSTEM” filed Jan. 27, 2023, which is incorporated by reference in its entirety herein for all purposes.

### BACKGROUND

Biomanufacturing is a technology that utilizes biological systems to produce commercially important biomaterials and biomolecules for use in medicines, food and beverage processing, and industrial applications. Biomanufacturing products may be generated from cultures of microbes, animal cells, or plant cells grown in specialized equipment. The cultured seeds used during production may be naturally occurring or derived using genetic engineering techniques. When using one or more bioreactors, one may develop automated processes that assist biotech companies optimize manufacturing processes and cultivate facilitating products to market faster. Typical biomanufacturing processes can be very time consuming and require a large amount of manual handling by a trained individual in a contaminant-free environment. For example, seed preparation and sample prep and analysis may also require individualized attention in addition to the bioreactions. Such techniques can be particularly burdensome for an experiment that may benefit from high throughput biomanufacturing.

Typical experiments may be governed by a recipe that provides specific instructions for guiding the experiment. A recipe will often have specific parameters such as ingredients, process instructions (e.g., temperature schedule, agitation schedule, and the like) and other guidance so that a bioreactor may be automatically controlled according to the parameters of the recipe. To these ends, a recipe may be altered and cultivated over time or may be subject to charge according to the desires of a user conducting each experiment. As such, recipes are often edited from experiment to experiment and also often edited during an experiment. A problem arises, however, that different edits may be made to recipes stored in different data stores by different controllers/users. Thus, competing recipe edits may be at odds with each other and each bioreactor may follow disparate and conflicting instructions. What is needed is a one-stop data store for storing a unique credentialed copy of the one and only recipe that will control an experiment at any given time.

### BRIEF DESCRIPTION OF THE DRAWINGS

Embodiments of the subject matter disclosed herein in accordance with the present disclosure will be described with reference to the drawings, in which:

FIG. 1 is a diagram of a biomanufacturing system utilized by customers via a client-facing data assimilation system according to an embodiment of the subject matter disclosed herein;

FIG. 2 is a diagram of computer system for facilitating computer communications in support of the client-facing data assimilation system according to an embodiment of the subject matter disclosed herein;

FIGS. 3-7 are screenshots of displays used at stages of the presentation, manipulation, and intake of data in the context of the client-facing data assimilation system according to embodiments of the subject matter disclosed herein

FIG. 8 is a flow chart of a method for presenting and facilitating the client-facing data assimilation system according to an embodiment of the subject matter disclosed herein; and

FIG. 9 is a diagram illustrating elements or components that may be present in a computer device or system configured to implement a method, process, function, or operation in accordance with an embodiment of the subject matter disclosed herein.

Note that the same numbers are used throughout the disclosure and figures to reference like components and features.

### DETAILED DESCRIPTION

The subject matter of embodiments disclosed herein is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

By way of an overview, the systems and methods described herein provide a data presentation, assimilation and monitoring system for a biomanufacturing system utilizing an array of bioreactors for conducting biomanufacturing experiments. In embodiments, the experiments may be controlled by recipes that can be created and altered by credentialed users. During experiments, credentialed users may monitor operational parameters of the bioreactors conducting experiments as well as batch and group parameters across multiple bioreactors. Further, credentialed users may alter recipes to the exclusion of other credentialed users while editing. Further yet, specific checklists may be utilized to ensure that correct and suited equipment (such as bioreactors and experiment bays) are matched with an experiment plan.

Embodiments will be described more fully hereinafter with reference to the accompanying drawings, which form a part hereof, and which show, by way of illustration, exemplary embodiments by which the systems and methods described herein may be practiced. This systems and methods may, however, be embodied in many different forms and should not be construed as limited to the embodiments set forth herein; rather, these embodiments are provided so that this disclosure will satisfy the statutory requirements and convey the scope of the subject matter to those skilled in the art.

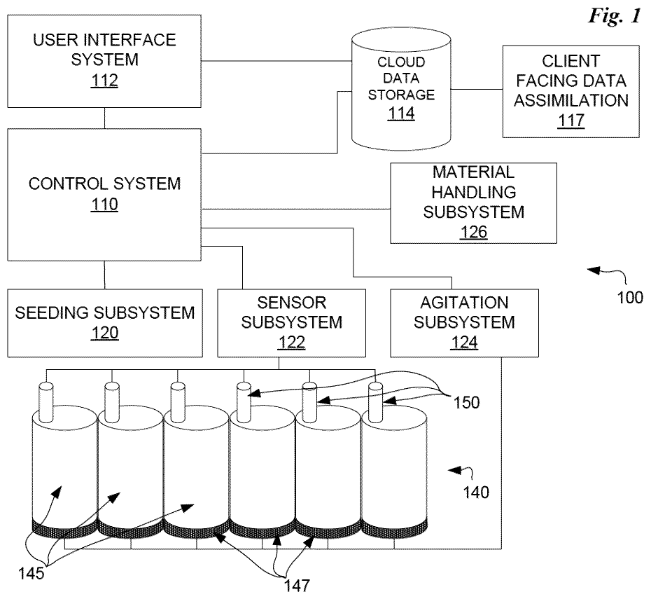

FIG. 1 is a diagram of a biomanufacturing system utilized by customers via a client-facing data assimilation system according to an embodiment of the subject matter disclosed herein. Various aspects of the biomanufacturing system 100 described herein may be applied to any of the particular applications set forth below. The system includes several functional blocks and devices that may include subsystems that utilize one or more processors for automation and tracking. The biomanufacturing system 100 may utilize an array of bioreactors 140 (sometimes called work cells) or may be implemented using a single bioreactor. Whether an array or a single bioreactor 140, the biomanufacturing system 100 may include additional subsystems for bioreactor control, data collection and data analysis as will be described below. It shall be understood that different aspects of the biomanufacturing system 100 can be appreciated individually, collectively or in combination with each other.

Biomanufacturing processes can be used for many applications. For instance, biomanufacturing can be utilized for production of biomass (e.g., viable cellular material), production of extracellular metabolites (e.g., chemical compounds), production of intracellular components (e.g., enzymes and other proteins), or transformation of a substrate (e.g., the substrate itself may be a product). Biomanufacturing processes are useful for biological experiments, drug manufacturing, food industry, biofuels, or many other applications. In some instances, it may be desirable to provide automated biomanufacturing systems and methods that allow for low risk of contamination, high levels of accuracy and repeatability, high throughput, controlled variations, quicker turnaround, and/or require less manpower.

FIG. 1 shows a block diagram of an overall biomanufacturing system 100 that may include an automated seeding subsystem 120, a bioreactor sensor subsystem 121, a bioreactor agitation subsystem 124, and/or a material handling subsystem 126. Each of these subsystems may be communicatively coupled to a control system 110 that is associated with a user-interface system 112 and a cloud-based database 114. The biomanufacturing system 100 may optionally include an enclosure (not shown). The enclosure may comprise a sterile enclosure that prevents contaminants from coming inside the enclosure. The area within the biomanufacturing system 100 is a clean, sterile environment, thereby reducing the risk of contamination to the experiments. The enclosure may partially or completely enclose each subsystem of the biomanufacturing system 100. The enclosure may be substantially fluid-tight and may be airtight and/or liquid-tight. In some embodiments, multiple enclosures may be provided. One or more stations or components may be provided in separate enclosures.

The bioreactor array 140 may comprise multiple bioreactors 145 that each include one or more respective individual sensor probes 150 for monitoring conditions of the environment inside each bioreactor 145 as well as individual agitators 147 for provides agitation for each bioreactor 145. Each vessel (e.g., each bioreactor) may be associated with multiple probes (not shown) each configured to determine a specific characteristic of the solution in the vessel such as pH level, temperature via a thermocouple (or the like), and dissolved oxygen (DO) levels. In some embodiments, these characteristics may be determined by a single probe configured to determine pH levels, temperature and DO levels simultaneously, or any combination of two or more characteristics. In still further embodiments, combinations of different characteristics may be determined by one probe with multiple characteristic sensing capabilities. Further, each probe may be communicatively coupled to the sensor subsystem 122 and each agitator 147 may be communicatively coupled to the agitation subsystem 124. Collectively, these subsystems may be under the control and direction of the control system 110 whereupon a user may utilize the user interface system 112 to control and direct the activities and procedures of the overall biomanufacturing system 100. Further, data about the control, state, and productions of the biomanufacturing system 100 may be communicated and stored in a cloud-based data store 114.

One or more processes within the biomanufacturing system 100 may be fully automated. That is, any process may be automated and executed without requiring human intervention. Further, such processes may be automated with the aid of one or more processors embodied in one or more computer systems. In some embodiments, transfer of materials from a seeding subsystem 120 to the bioreactor array 140 may be fully automated. In some embodiments, transfer of materials from the bioreactor array 140 to the material-handling subsystem 124 may be fully automated. In some embodiments, one or more robotic components may aid in the automated processes. In some cases, one or more robotic components may comprise one or more robotic arms or other robotic components such as gantry.

As discussed previously, the biomanufacturing system 100 may comprise one or more bioreactors 145 within a bioreactor array 140. Each bioreactor may correspond to an experiment. Each bioreactor 145 may correspond to an individual biomanufacturing process, which may be associated with a unique, individual experiment. Each bioreactor 145 may be independently operating and/or controllable relative to another bioreactor in the bioreactor array 140. Any number or arrangement of bioreactors 145 may be provided, as provided in greater detail elsewhere herein. Further yet, one or more modular components may be provided in the biomanufacturing system 100. For instance, components, such as bioreactors 145 and/or other equipment (e.g., sensor probes, agitators 147, and the like) may be swapped in or out as needed.

Each individual bioreactor 145 may be seeded with input strains from the seeding subsystem 120. For strain input, a strain or set of strains may be provided to the bioreactor 145. Any description herein of providing an input strain may be applied to providing a set of input strains or multiple input strains. An input strain may be provided via one or more tubes, frozen stocks, plates, beads, wells, channels, or any other technique. The input strain may be provided manually or may be loaded in an automated fashion. In some embodiments, the bioreactor 145 may be capable of accommodating multiple types of strain inputs. For example, the bioreactor 145 may be able to accept one or more tubes, and/or one or more plates with an input strain or set of input strains. Similarly, each individual bioreactor 145 may be populated with media from the seeding subsystem 120. Any description herein of providing media may also be applied to providing a set of input strains or multiple input strains or vice versa.

As experiments are controlled and conducted within the biomanufacturing system 100, all relevant data may be stored in the cloud data storage 114. The cloud data storage 114 may also be communicatively coupled to a client-facing data assimilation system 117. The client-facing data assimilation system may be one or more computer systems that collectively present data from the cloud data storage to a credentialed user (e.g., an owner of the underlying experiment(s) in the biomanufacturing system). The client-facing data assimilation system 117 is discussed in more detail in the following paragraphs.

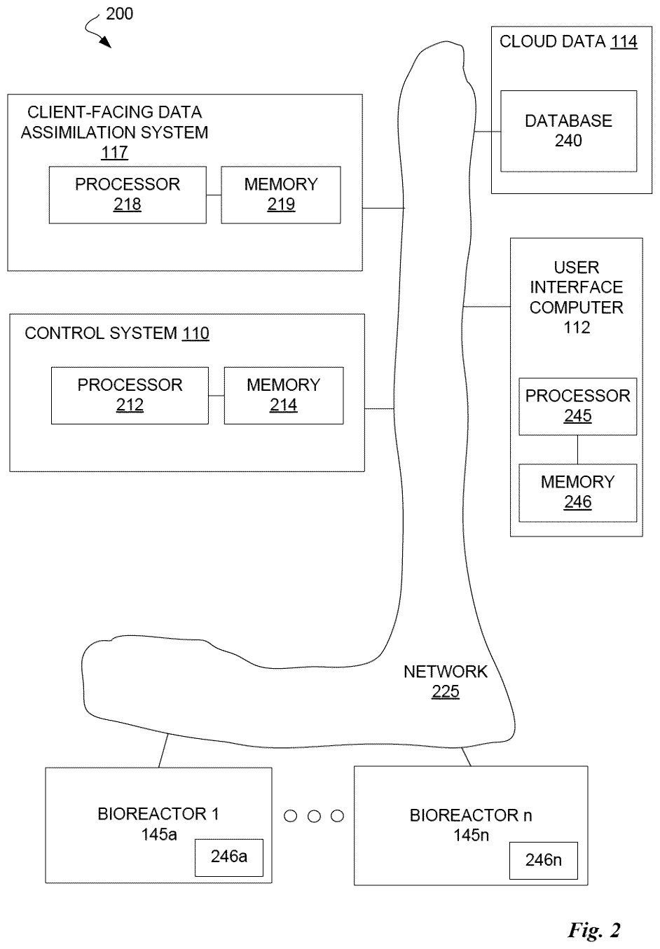

FIG. 2 is a diagram of computer system 200 for facilitating computer communications in support of the client-facing data assimilation system 117 according to an embodiment of the subject matter disclosed herein. The various systems and computers described with respect to FIG. 2 may be stand-alone computer systems or may be part of an overall computing system and/or one or more cloud-based computing systems. As such, each of the individual computer systems with FIG. 2 may be communicatively coupled to each other through a computer network 225 such as the Internet.

The overall system 200 may be ultimately subject to some control though use of a control system 110 as depicted in FIG. 1. In the system 200 of FIG. 2, the control system 110 may also include several dedicated components such as a processor 212 and local memory 214. The control computer system 110 may execute instructions stored in the local memory 214 via processor 212 to achieve goals of the client-facing data assimilation system, though not necessarily under direct control. The control computer system 110 may be configured to retrieve and implement instructions to control one or more bioreactors according to one or more recipes.

The overall system 200 further includes several bioreactor vessels in a bioreactor array that includes, as shown, at least bioreactors 145a-145n that respectively may include dedicated processors 146a-146n for handling local bioreactor control and communications. As mentioned above, control computer system 110 may be configured to execute instructions to control each of these bioreactors 145a-145n.

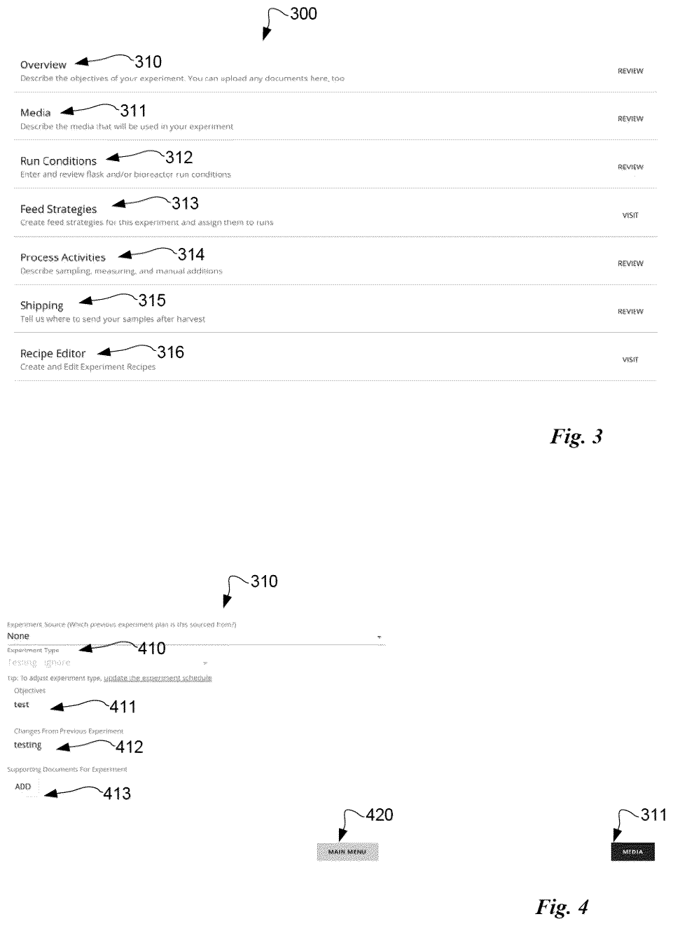

The overall system 200 may also include the afore-mentioned user-interface computer system 112 that also may include several dedicated components such as a processor 245 and local memory 246 for executing local control instructions stored within the local memory 246. The overall system 200 may also include the afore-mentioned cloud data storage 114 that may have a dedicated database 240 that is a repository of client data and client recipes. Each recipe stored therein may be associated with a specific credentialed user of the client-facing data assimilation system 117. Further yet, the client-facing data assimilation system 117 may also include a dedicated processor 218 configured to execute local instructions stored on a dedicated memory 219 at the client-facing data assimilation system 117. The client-facing data assimilation system 117 may be configured to facilitate credentialed user logins and lock-out editing control of the database 240 in the cloud data storage 114. Each of these systems may be communicatively coupled to all other systems via the computer network 225. Together, these computer systems may assist in facilitating a user experience for controlling experiments and data collected and presented as illustrated in the screenshots of FIGS. 3-7 discussed next.

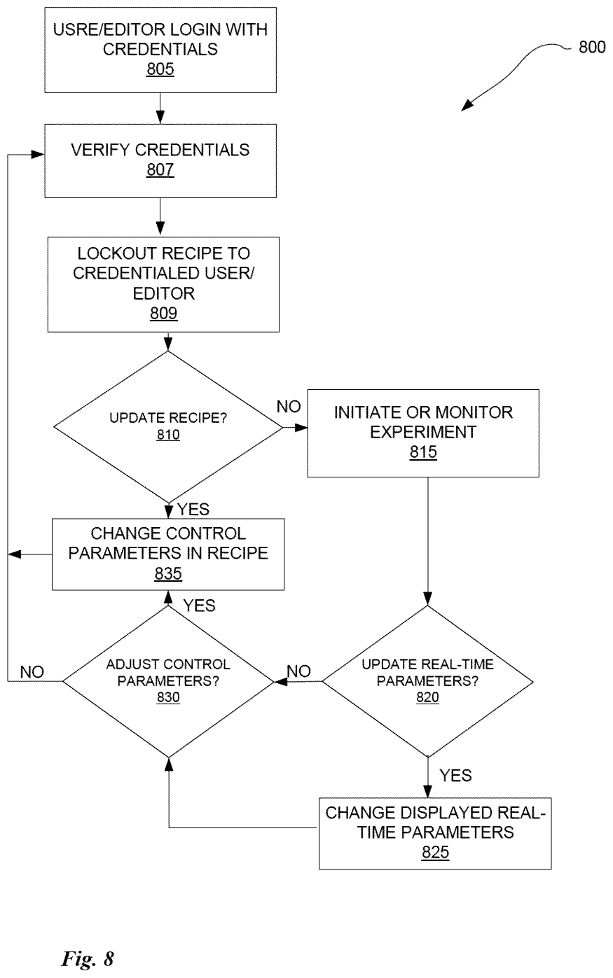

FIGS. 3-7 are screenshots of displays used at stages of the presentation, manipulation, and intake of data in the context of the client-facing data assimilation system 117 according to embodiments of the subject matter disclosed herein. The screenshots presented are merely illustrative examples of the kinds of data that may be presented to a logged-in user/editor. Other data and presentations of data may be utilized, but the examples illustrated here provide a detailed example of one set of ways to interface with user/editor. The method discussed below with respect to FIG. 8 also provides illustrative examples of one manner of facilitating data assimilation, manipulation, and display in the context of the subject matter disclosed herein.

Turning to FIG. 3, a screenshot of an overall client-facing menu 300 is shown. This menu 300 may be shown on a per-experiment basis. That is, each of the options discussed next for this menu 300 corresponds to one and only one experiment associated with a specific credentialed client. This menu may be shown to a client once a client accesses the client-facing data assimilation system 117 and passes a credentials test (e.g., username and password, or the like). The menus include several top-level options for navigation including an overview page 310, a media page 311, a run conditions page 312, a feed strategies page 313, a process activities page 314, a shipping page 315, and a recipe editor page 316.

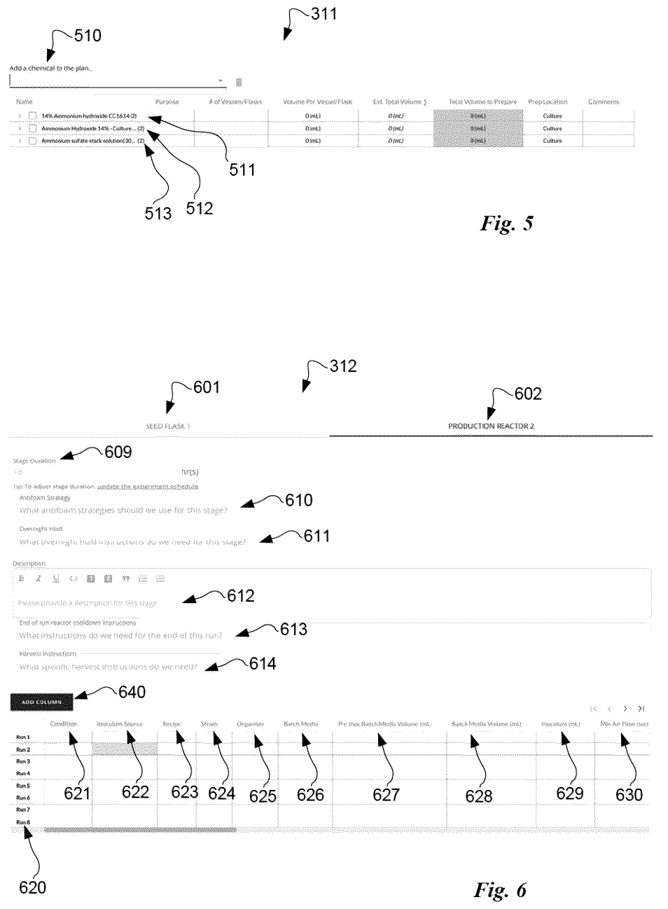

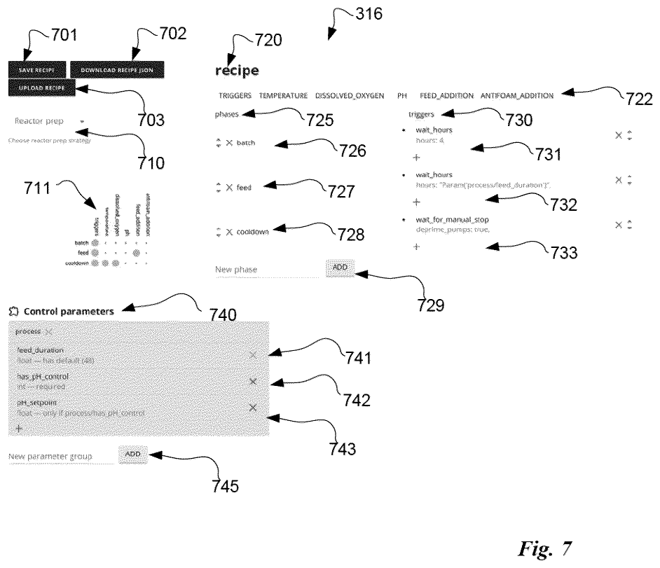

As a general level of description, the overview page 310 may navigate to a displayed page showing objectives and documentation for the respective experiment. Further, the media page 311 (described below with respect to FIG. 4) may navigate to a displayed page showing the specific media that is to be used for the respective experiment. Further yet, the run conditions page 312 (described below with respect to FIG. 5) may navigate to a displayed page showing flask and/or bioreactor run conditions (e.g., temperature controls, agitation schedules, and the like). The feed strategies page 313 (described below with respect to FIG. 6) may navigate to a displayed page showing specific parameters of the feed strategy for this experiment. The process activities page 314 may navigate to a displayed page showing specific parameters for sampling, measuring, and adjusting this specific experiment. Each of these afore-mentioned pages may also display real-time data collected from probes and samples of the experiment as it is running. Lastly, a user may navigate to a shipping page 315 to see where concluded experiments are to be sent as well as a recipe editor page 316 (described below with respect to FIG. 7) to adjust, edit or otherwise alter any parameter of the experiment in real-time. Some of these specific pages are described next in the following paragraphs.

FIG. 4 is a screenshot of an overview page 310 that may be navigated to by a client from the initial menu of FIG. 3. This page 310 may be shown on a per-experiment basis and describe any overview descriptions and parameters of the respective experiment. For example, a client may choose to store general descriptions and data on this page including experiment type 410, experiment objectives 411, and changes from any previous experiment 412. Further a client may choose to upload additional documentation using an add button 413. This allows a client to store any document (e.g., text documents, image documents, and the like) in the cloud storage with a repetitive experiment. This overview page 301 also provides for navigation buttons to return to the main menu 420 or to proceed to the media page 311. Additional navigation and documentation possibilities are contemplated but not shown here for brevity.

FIG. 5 is a screenshot of a media description page 311 that may be navigated to by a client from the initial menu of FIG. 3. This page 311 may be shown on a per-experiment basis and describe the composition of the specific media used in the respective experiment. In one embodiment, this page 311 merely shows the specific composition of the media that is being used (or is to be used) in the respective experiment. For example, this specific experiment shows that the media is composed of 14% ammonium hydroxide 511, ammonium hydroxide 14% culture 512, and ammonium sulfate stock solution 513. In other embodiments, such as the one shown here, a user/editor may utilize an add button 510 to add additional chemical composition to the media recipe as shown. In turn, such additions to this page may also alter the overall recipe as stored in the cloud data storage. Such a change here will also be reflected in the recipe displayed with respect to page 316 as will be described below.

FIG. 6 is a screenshot of a run conditions page 312 that may be navigated to by a client from the initial menu of FIG. 3. This page 312 may be shown on a per-experiment basis and describe specific run conditions used in the respective experiment. The overall display may be bifurcated into a flask section display 601 or a bioreactor section display 602. In this screenshot 312, the bioreactor section display 602 is shown. Here, several specific run conditions for a bioreactor are shown. These parameters as shown may represent real-time conditions of the experiment as determined by probes, sensors and/or samples taken in real-time or near-real time (e.g., every 2 minutes or some other suitable time period).

In this bioreactor run conditions display, several parameters are displayed as drawn from the recipe and/or real-time probes and sensors. These data points include a stage duration 609 indicating a time period for the current stage of the experiment. Further, this page shows any antifoam strategies 610 and/or overnight hold strategies 611 that have been assigned to this experiment. Additional data points displayed may include a description 612 of the current stage of the experiment, end-of-run reactor cooldown instructions 613, and harvest instructions 614. Further yet, specific run conditions may be assimilated and displayed here in a run condition list 620. Here, a user may enter run conditions that ultimately alter the stored recipe or may simply reflect what has already been stored with the displayed recipe. Each run condition may include several parameters such as condition name 621, inoculum source 622, recipe ID 623, strain name 624, organism 625, batch media, 626, pre-inoculation batch media volume 627, batch media volume 628, inoculum 629 and maximum air flow 630. As these column headings may be associated with specific experiment some, all or none of these columns may be present in any display of specific run conditions and a user/editor may be able to add columns using a functional add column button 640.

FIG. 7 is a screenshot of a recipe editor page 316 that may be navigated to by a client from the initial menu of FIG. 3. This page 316 may be shown on a per-experiment basis and allow functionality to a client for changing, adjusting, or otherwise altering specific parameters for the recipe for the respective experiment. Further, the current display of the parameters of the recipe may be shown in the specific parameter locations on this page 316. In this manner, a logged-in user/editor may change specific parameters in a specific recipe here and then persist the changes to the cloud data storage. Thus, this one-and-only editing location may ensure than changes to any recipes are handled in a serial manner so as to avoid conflicting changes or version control issues as only one user/editor may have editable control of any recipe at any given time. This editable version control schema is discussed in greater detail below with respect to FIG. 8. For now, the remainder of the discussion for FIG. 7 is focused on which specific parameters may be adjusted, changed, or altered on this page 316.

In this page view 316, a user/editor may have three specific recipe control options in the form of actionable buttons. A user/editor may download a recipe from the cloud data storage by actuating the download recipe button 702. This will populate all relevant fields with currently stored parameters for the selected recipe. Similarly, a user/editor may upload the currently displayed recipe to the cloud data storage by actuating the upload recipe button 703. This action will then replace any changed or altered parameters for the given displayed recipe in the cloud data storage. Further, as a user/editor makes changes to displayed parameters of the recipe, the user/editor may press the save recipe button 701 to save a local version of the recipe before uploading to the cloud data storage. In this manner, the user/editor may make multiple recipe parameter edits, perhaps saving locally after each change, prior to uploading the recipe changes to the cloud data storage. In addition to these three specific recipe control options, a user/editor may select a specific reactor preparation strategy at actionable button 710.

In this recipe editor page view 316, the overall recipe and several parameters may be shown in a recipe display 720. This recipe display 720 may also include a recipe name (instead of simply displaying the word “recipe”). The recipe displayed will typically include a number of phases 725 as well as a number of triggers 730. In this example recipe, there are three phases shown that include a batch phase 726, a feed phase 727, and cooldown phase 728. Each of the phases may include additional information (e.g., parameters) for the specific direction associated with each phase. Further, a user/editor may add phases to the recipe using the add phase button 729.

The recipe display 720 may also include additional information in the display box. In this example, there is also a display of the triggers 730 associated with recipe. The example triggers shown here include a first wait threshold trigger 731, a second wait threshold trigger phase 732, and manual stop trigger 733. The recipe display may also show several other parameters and a user/editor may control what is displayed in the display box. This is generally accomplished through a parameter menu 722 wherein a user/editor may choose to display specific parameter groups such as trigger parameters, temperature parameters, dissolved oxygen parameters, pH parameters, feed addition parameters, and antifoam parameters. So that a user/editor may quickly determine which parameters may be part of a recipe, a parameter matrix 711 may be displayed with indications of types of parameters in a table format.

The recipe editor page view 316 may also show a control parameter editor display 740 wherein a user/editor may select a specific control parameter from the recipe display 720 to look at in greater detail. As such, there are three specific control parameters shown in this example whereby a user/editor may alter, change, or adjust the specific parameters of feed duration parameter 741, a pH control parameter 742 and pH setpoint parameter 743. A user/editor may also add or remove parameters to this group or may add entirely new parameters groups using action button 745. All of the control parameters that may be changed, adjusted, or altered as discussed herein may be done so within the context of a method involving aspects of the overall client-facing data assimilation system as discussed next in FIG. 8.

FIG. 8 is a flow chart of a method 800 for presenting and facilitating the client-facing data assimilation system according to an embodiment of the subject matter disclosed herein. In this method, experiments may already have been initiated or may be waiting to be initiated. The method covers the process for updating, changing, and/or altering recipes stored in a cloud data store whether or not an experiment has been initiated. Thus, a skilled artisan understands that the order of the steps as presented may be different that the example method 800 as shown depending on how and when various experiments corresponding to recipes have been or still being conducted.

Thus, in this embodiment, a user/editor login may access the client-facing data assimilation system with login credentials at step 805. At step 807, the client-facing data assimilation system may then verify the login credentials to allow or disallow access. Assuming verified login credentials, the user/editor may then select a specific recipe for monitoring or updating, at step 809, whereupon the specific recipe is locked out the user/editor to ensure that no other credentialed user may simultaneously attempt to make recipe changes. That is, the plurality of other remote computer systems associated with other would-be users/editors prevents other simultaneous editing as the lockout provides for one and only one remote computer system to alter the current version of the recipe. In other embodiments, other users (credentialed or otherwise) may be able to monitor experiment via a recipe display but will still remain locked out from making any edits or changes to the recipe as stored in the cloud data storage.

When a credentialed user is logged in and has a specific recipe locked out, the system continuously monitors for any changes or alterations to the recipe as entered by the user/editor at query step 810. Such a query may be forced by actions of the user/editor (e.g., the upload recipe button of FIG. 7 is pressed) or may be on a looped query time, such as every 30 seconds while a recipe is locked out. Simultaneous to this specific query 810, an experiment utilizing the locked-out recipe may be initiated or otherwise continue after having been previously initiated at step 815. That is, if there is no specific update that would result in a “yes” answer to query step 810, then an associated experiment may either continue or be initiated at step 815. However, if the answer to query step 810 is “yes” then the method will change the recipe stored in the cloud data storage at step 835. After a recipe update, the method may loop back to the verify credentials step 807 in order to determine if the recipe should remain locked out.

As experiments are initiated or ongoing, the method 800 may also monitor for any specific parameters to monitor in real-time for changing a display of current conditions at query step 820. If there are parameters to update (e.g., sensors and/or probes having updated readings that may impact the experiment or otherwise be in need of updating a real-time display of the experiment, then the method moves to an update real-time display step at 825. If there are no updates or after current updates, the method moves to query step 830 whereby any updated parameters may trigger a change in the recipe itself. If yes, then the recipe is once again changed at step 835. In any case, the entire method still loops back to verifying credentials again as all queries are repeated until the recipe lockout is relinquished.

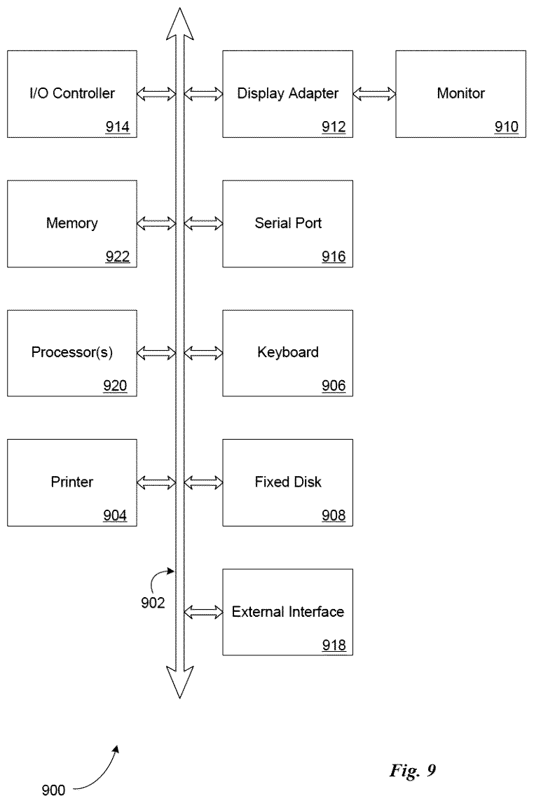

FIG. 9 is a diagram illustrating elements or components that may be present in a computer device or system configured to implement a method, process, function, or operation in accordance with an embodiment. In accordance with one or more embodiments, the system, apparatus, methods, processes, functions, and/or operations for enabling efficient configuration and presentation of a user interface to a user based on the user's previous behavior may be wholly or partially implemented in the form of a set of instructions executed by one or more programmed computer processors such as a master control unit (MCU), central processing unit (CPU), or microprocessor. Such processors may be incorporated in an apparatus, server, client or other computing or data processing device operated by, or in communication with, other components of the system. As an example, FIG. 9 is a diagram illustrating elements or components that may be present in a computer device or system 900 configured to implement a method, process, function, or operation in accordance with an embodiment. The subsystems shown in FIG. 9 are interconnected via a system bus 902. Additional subsystems include a printer 904, a keyboard 906, a fixed disk 908, and a monitor 910, which is coupled to a display adapter 912. Peripherals and input/output (I/O) devices, which couple to an I/O controller 914, can be connected to the computer system by any number of means known in the art, such as a serial port 916. For example, the serial port 916 or an external interface 918 can be utilized to connect the computer device 900 to further devices and/or systems not shown in FIG. 9 including a wide area network such as the Internet, a mouse input device, and/or a scanner. The interconnection via the system bus 902 allows one or more processors 920 to communicate with each subsystem and to control the execution of instructions that may be stored in a system memory 922 and/or the fixed disk 908, as well as the exchange of information between subsystems. The system memory 922 and/or the fixed disk 908 may embody a tangible computer-readable medium.

The present disclosures as described above can be implemented in the form of control logic using computer software in a modular or integrated manner. Based on the disclosure and teachings provided herein, a person of ordinary skill in the art will know and appreciate other ways and/or methods to implement the present disclosure using hardware and a combination of hardware and software.

Any of the software components, processes or functions described in this application may be implemented as software code to be executed by a processor using any suitable computer language such as, for example, Assembly language Java, JavaScript, C, C++, or Perl using, for example, conventional or object-oriented techniques. The software code may be stored as a series of instructions, or commands on a computer readable medium, such as a random-access memory (RAM), a read only memory (ROM), a magnetic medium such as a hard-drive or a floppy disk, or an optical medium such as a CD-ROM. Any such computer readable medium may reside on or within a single computational apparatus and may be present on or within different computational apparatuses within a system or network.

All references, including publications, patent applications, and patents, cited herein are hereby incorporated by reference to the same extent as if each reference were individually and specifically indicated to be incorporated by reference and/or were set forth in its entirety herein.

The use of the terms “a” and “an” and “the” and similar referents in the specification and in the following claims are to be construed to cover both the singular and the plural, unless otherwise indicated herein or clearly contradicted by context. The terms “having,” “including,” “containing” and similar referents in the specification and in the following claims are to be construed as open-ended terms (e.g., meaning “including, but not limited to,”) unless otherwise noted. Recitation of ranges of values herein are merely indented to serve as a shorthand method of referring individually to each separate value inclusively falling within the range, unless otherwise indicated herein, and each separate value is incorporated into the specification as if it were individually recited herein. All methods described herein can be performed in any suitable order unless otherwise indicated herein or clearly contradicted by context. The use of any and all examples, or exemplary language (e.g., “such as”) provided herein, is intended merely to better illuminate embodiments, and does not pose a limitation to the scope of the disclosure unless otherwise claimed. No language in the specification should be construed as indicating any non-claimed element as essential to each embodiment of the present disclosure.

Different arrangements of the components depicted in the drawings or described above, as well as components and steps not shown or described are possible. Similarly, some features and sub-combinations are useful and may be employed without reference to other features and sub-combinations. Embodiments have been described for illustrative and not restrictive purposes, and alternative embodiments will become apparent to readers of this patent. Accordingly, the present subject matter is not limited to the embodiments described above or depicted in the drawings, and various embodiments and modifications can be made without departing from the scope of the claims below.

What is claimed is:

1. A system for maintaining a single source for biomanufacturing system operational instructions, the system comprising:
a biomanufacturing system having a bioreactor configured to conduct an experiment according to recipe instructions;
a processor communicatively coupled to the biomanufacturing system and configured to execute the recipe instructions to operate the bioreactor;
a data store communicatively coupled to the processor and configured to store a current version of the recipe instructions, the current version of the recipe instructions including executable instructions for operation of the bioreactor; and
a plurality of remote computer systems each configured to have access the data store with credentials, the credentials allowing for one and only one remote computer system to lockout the current version for exclude any other of the plurality of remote computer systems and configured to alter the current version to the exclusion of any other remote computer system while the current version is locked out;
wherein the processor executes instructions corresponding to the current version of the recipe instructions stored in the data store at the time of execution of the instructions.

2. The system of claim 1, wherein the biomanufacturing system further comprises a plurality of bioreactors, each bioreactor associated with a respective current version of a set of recipe instructions stored in the data store.

3. The system of claim 1, wherein the current version of the recipe instructions corresponds to a latest version of the recipe instructions stored in the data store.

4. The system of claim 1, wherein the processor, the data store, and the plurality of remote computers are coupled to each other via a computer network.

5. The system of claim 1, wherein the current version of the recipe instructions further comprises instructions for controlling operational parameters of the bioreactor including one or more of the group comprised of: pH target level, temperature target level, dissolved oxygen target level, agitation level, feed level, density target level; weight target level; and time period.

6. The system of claim 1, wherein the current version of the recipe instructions further comprises instructions for controlling operational parameters of the bioreactor including one or more of the group comprised of: a batch phase, a feed phase, a cooldown phase, an agitation phase, and a heating phase.

7. The system of claim 1, further comprising an agitation subsystem coupled to the bioreactor and configured to operate according to the current version of the recipe instructions.

8. The system of claim 1, further comprising a materials handling subsystem coupled to the bioreactor and configured to operate according to the current version of the recipe instructions.

9. The system of claim 1, further comprising a seeding subsystem coupled to the bioreactor and configured to operate according to the current version of the recipe instructions.

10. The system of claim 1, further comprising a sensor subsystem coupled to the bioreactor and configured to operate according to the current version of the recipe instructions.

11. The system of claim 1, further comprising a user-interface computer system communicatively coupled to the biomanufacturing system and configured to display operational parameters of the current version of the recipe without altering the current version of the recipe instructions.

12. A computer-based method for operating a biomanufacturing system, the method comprising:
establishing a recipe at a cloud-based data store that is part of a client-facing data assimilation system, the recipe having a plurality of operating parameters for operating a bioreactor in a biomanufacturing system;
accessing the client-facing data assimilation system from a remote client computer;
verifying credentials to lock out the recipe so that no other remote client computer may access the recipe while the recipe is locked out to the remote client computer having verified credentials;
changing one or more operating parameters of the locked-out recipe to establish an updated recipe; and
uploading the updated recipe with the one or more changed operating parameters to a cloud-based data store.

13. The method of claim 12, wherein the accessing the client-facing data assimilation system from a remote client computer further comprises accessing the client-facing data assimilation system via a computer network.

14. The method of claim 12, wherein establishing the recipe further comprises storing instructions for controlling operating parameters of the bioreactor including one or more of the group comprised of: pH target level, temperature target level, dissolved oxygen target level, agitation level, feed level, density target level; weight target level; and time period.

15. The method of claim 12, wherein establishing the recipe further comprises storing instructions for controlling operating parameters of the bioreactor including one or more of the group comprised of: a batch phase, a feed phase, a cooldown phase, an agitation phase, and a heating phase.

16. The method of claim 12, further comprising updating a display of operating parameters in response to uploading the updated recipe.

17. The method of claim 12, further comprising unlocking the recipe in response to uploading the updated recipe.

18. The method of claim 12, further comprising saving changed operating parameters to a local data store prior to uploading the updated recipe.

19. The method of claim 12, further comprising displaying a media composition of the recipe on a display of the remote client computer.

20. The method of claim 12, further comprising displaying editable fields of the operating parameters the recipe prior to changing one or more operating parameter.
